SEQUENCE LISTING (6 sequences)

SEQ ID NO: 1 - protein, 6 aa, Artificial Sequence
  Peptide conjugated with Hyaluronic aicd derivative
WRYMVM

SEQ ID NO: 2 - protein, 6 aa, Artificial Sequence
  Peptide conjugated with Hyaluronic acid derivative
WRWWWW

SEQ ID NO: 3 - protein, 6 aa, Artificial Sequence
  Peptide conjugated with Hyaluronic acid derivative
WKYMVM

SEQ ID NO: 4 - protein, 6 aa, Artificial Sequence
  Peptide conjugated with Hyaluronic acid derivative
WWRWWM

SEQ ID NO: 5 - protein, 7 aa, Artificial Sequence
  Peptide conjugated with Hyaluronic aicd derivative
CWRYMVM

SEQ ID NO: 6 - protein, 7 aa, Artificial Sequence
  Peptide conjugated with Hyaluronic acid derivative
CWRWWWW

(19) United States
(12) Patent Application Publication
(10) Pub. No.: US 2010/0210509 A1
(43) Pub. Date: Aug. 19, 2010

(54) LONG ACTING HYALURONIC ACID - PEPTIDE CONJUGATE

(75) Inventors: Eun-Ju Oh, Busan (KR); Jung-Wook Kim, Seoul (KR); Sung-Ho Ryu, Pohang-city (KR); Sei-Kwang Hahn, Pohang-city (KR)

Correspondence Address: LEXYOUME IP GROUP, LLC, 5180 PARKSTONE DRIVE, SUITE 175, CHANTILLY, VA, 20151, US

(73) Assignees: Postech Academy-Industry Foundation, Pohang-city (KR); Posco, Pohang-shi (KR)

(21) Appl. No.: 12/680,955

(22) PCT Filed: Oct. 9, 2008
(86) PCT No.: PCT/KR08/05943

§ 371 Date: Mar. 31, 2010

Related U.S. Application Data
(60) Provisional application No. 60/978,566, filed on Oct. 9, 2007.

(51) Int. Cl. A61K 38/16; C07K 9/00

(52) U.S. Cl. 514/8; 530/322

(57) ABSTRACT

The invention relates to a novel bioconjugation protocol for peptide suitable for in vivo applications. Bioconjugation of the peptide to HA derivative increases its half life in circulation contributing for a high efficacy. More over, conjugate of HA derivative and peptide which is treated with hyaluronidase shows increased bioactivity. And also, in contrast to PEGylation, HA derivative can be conjugated with many numbers of peptide molecules per single HA derivative chain, which enables multiple action of peptide drugs.

### BACKGROUND OF THE INVENTION

(a) Field of the Invention

The invention relates to a long-acting formulation of biopharmaceuticals, more specifically a long acting conjugate of peptide with HA derivative having a long-term stability and a high efficacy, thereby improving patient compliance and quality of medical services. The invention provides a method of preparing a conjugate of peptide with HA derivative comprising the step of synthesizing HA derivative and conjugating HA derivative with peptide.

(b) Description of the Related Art

Conjugation of therapeutic protein or peptide sequence with biodegradable polymer can prolong the maintenance of therapeutic drug levels relative to administration of the drug itself. Sustained release may be extended up to several weeks depending on the formulation and the active ingredient conjugated. However, many active ingredients, especially therapeutic protein or peptide sequence such as an agonistic or antagonistic peptide for inflammatory disease associated with formyl peptide receptor like 1 (FPRL1), are damaged or made unstable by the procedure required to conjugate or encapsulate the conjugate in the polymeric carriers. Furthermore, the charged, polar nature of many peptides may limit the extent of conjugated molecule of the peptide to the biodegradable polymer and may lead to rapid loss of a fraction of the conjugate when first administered.

The FPRL1 is one of the chemoattractant receptors encompassing G protein-coupled seven transmembrane domains. It is mostly expressed in phagocytic leukocyte and stimulates innate immunity, such as chemotactic migration, pro-inflammatory cytokine secretion and degranulation. When inflammation and infection occur, chemotactic factors bind to specific heterotrimeric G protein-coupled receptors (GPCRs) such as FPRL1 receptor on the leukocyte surface. Activation of these receptors leads to directed migration, granule mobilization and activation of the nicotinamide adenine dinucleotide phosphate (NADPH) oxidase. The reactive oxygen species generated by the oxidase are important for microbial killing and for intercellular signaling in astrocytoma cell lines, neutrophils, monocytes, T cells and HUVECs.

Several agonistic and antagonistic peptide sequences for FPRL1 receptor have been investigated as drug candidates for inflammatory diseases such as sepsis, asthma and rheumatoid arthritis. Trp-Lys-Tyr-Met-Val-DMet (WKYMVm, SEQ ID NO: 3) is one of the widely studied agonist peptides that selectively binds to and activates FPRL1 receptor in FPRL1 over-expressing RBL-2H3 cells. Trp-Arg-Trp-Trp-Trp-Trp (WRWWWW, SEQ ID NO: 2) is an FPRL1 antagonist peptide inhibiting the binding of agonists to the specific receptor, FPRL1.

Recently, the bioconjugation technology using synthetic and natural polymers like poly(ethylene glycol) (PEG) and hyaluronic acid(HA) has been widely used for the development of various biopharmaceuticals with feasible pharmacokinetic characteristics. The chemical attachment of PEG to the biopharmaceuticals, such as protein and peptide drugs, has been reported to increase the drug efficacy by reducing renal clearance, decreasing the immuno-response and alleviating the enzymatic degradation in the body. There are several commercialized PEGylation products, such as Neulasta® (pegfilgrastim) by Amgen, Somavert® (pegvisomant) by Pfizer, PEGASYS® (peginterferon alfa-2a) by Roche and so on.

However, the negative effect of PEGylation has been also reported. A repeated injection of PEGylated liposomes has been reported to result in their diminished long-circulating characteristics by so called ‘accelerated blood clearance’ (ABC) phenomenon. In addition, PEGylation of glucagon like peptide-1 (GLP-1) has been reported to bring about a significant decrease in its cAMP activity. The branch-type PEGylation with a molecular weight of 43,000 Da caused even greater biological activity loss.

As an alternative to replace the roles of PEG, HA has been investigated as a novel drug carrier for various protein and peptide drugs (Kim et al., J. Control. Rel. 104, 323-335, 2005). HA is a natural linear polysaccharide composed of alternating disaccharide units of D-glucuronic acid and N-acetyl-D-glucosamine with β (1→4) interglycosidic linkage. HA is the only non-sulfated glycosaminoglycan (GAG) which is abundant in synovial fluid and extracellular matrix (ECM). The fact, that HA molecules from different sources have the same primary structure, explains the molecular basis for its natural biocompatibility. HA plays important roles in the regulation of cell behaviors including cell migration and proliferation, CD44, RHAMM, and LYVE-1 have been identified as HA receptors.

Because of the various biological functions and unique physico-chemical properties, HA and modified HA have been widely used for drug delivery, arthritis treatment (Kim et al., J. Control. Rel. 104, 323-335, 2005), ophthalmic surgery, and tissue engineering. Especially for drug delivery applications, HA with a high molecular weight over 2 million Da was used for the sustained release formulation of human growth hormone (Kim et al, J. Control. Rel. 104, 323-335, 2005) and selectively crosslinked HA hydrogels were used for the encapsulation of erythropoietin (Motokawa et al., J. Biomed. Mater. Res. 78A, 459-465, 2006). HA was also used for the conjugation with active cytotoxic agents, such as paclitaxel (Luo et al, Biomacromolecules 1, 208-218, 2000) and doxorubicin (Luo et al., Pharm. Res. 19, 396-402, 2002).

### SUMMARY OF THE INVENTION

The present invention provides a new long-acting formulation of biopharmaceuticals such as conjugate of peptide with HA derivative.

An object of the present invention is to provide a method of preparing a conjugate of peptide with HA derivative comprising the step of: synthesizing HA derivative and conjugating HA derivative with peptide.

Further object of the present invention is to provide a method of delivering a peptide comprising administering a conjugate of peptide with HA derivative to a subject in need.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 is a schematic drawing showing synthesis of HA-AEMA (a), and conjugate of CWRYMVm (SEQ ID NO: 5) with HA-AEMA (b).

FIG. 2 is ¹H NMR spectra HA-AEMA (a), conjugate of CWRYMVm (SEQ ID NO: 5) and HA-AEMA [The number of CWRYMVm (SEQ ID NO: 5) molecules per single HA-AEMA chain is 23] (b).

FIG. 3 is a Gel permeation chromatograms (GPC) of the peptide (CWRYMVm, SEQ ID NO: 5) at a right peak and conjugate of CWRYMVm (SEQ ID NO: 5) with HA-AEMA at a left peak detected at 280 nm.

FIG. 4 is a graph showing the content of peptide (CWRYMVm, SEQ ID NO: 5), the number of peptide molecules per single HA chain in conjugate of peptide with HA-AEMA, and the resulting bioconjugation efficiency(%).

FIG. 5 is a graph showing in vitro serum stability of peptide (CWRYMVm, SEQ ID NO: 5)() and conjugate of peptide with HA-AEMA in the fetal bovine serum(FBS, 50 vol %) solution. The number of peptide molecules per single HA chain was 5(◯), 19(▾), 33(□), respectively.

FIG. 6(a) is a Western blots for phospho-extracellular signal-regulated kinase (pERK) levels in the FPRL1 over-expressing RBL-2H3 cells after 5 minutes of stimulation with a control (no treatment), aminoethyl methacrylated hyaluronic acid (HA-AEMA), peptide (CWRYMVm, SEQ ID NO: 5), a mixture of peptide and HA-AEMA (HA+CWRYMVm), and three kind of conjugate of peptide with HA-AEMA samples (HA-CWRYMVm) with and without hyaluronidase (HAse) treatment. The numbers (5, 8, and 23) represent the number of peptide molecules per single HA chain in conjugate of peptide with HA-AEMA.

FIG. 6(b) is a graph showing Densitometry of Western blot bands for the samples in FIG. 6(a).

FIG. 7 is a Calcium fluorescence imaging of FPRL1 over-expressing RBL-2H3 cells after stimulation with (a) a control (no treatment) and (b) conjugate of peptide with HA-AEMA sample(HA-CWRYMVm) for FPRL1 receptor. The number of peptide molecules per single HA chain is 23.

FIG. 8 is a graph Densitometry of Western blot bands for phospho-extracellular signal-regulated kinase (pERK) levels in the FPRL1 over-expressing RBL-2H3 cells after 5 minutes of stimulation with a control (no treatment), peptide (CWRYMVm, SEQ ID NO: 5), and three kind of conjugate of peptide with HA-AMEA samples (HA-CWRYMVm) for FPRL1 receptor. The numbers of 5, 8, and 23 represent the number of peptide molecules per single HA chain in the conjugates. The results without co-treatment (first bars, dark cyan bands, WRWWWW−) were compared to those after co-treatment with antagonistic peptide of WRWWWW (SEQ ID NO: 2) for FPRL1 receptor (second bars, dark blue bands, WRWWWW+). The results without co-treatment are presented as means±S.D. of three independent experiments

### DETAILED DESCRIPTION OF THE PREFERRED EMBODIMENTS

These and other objects of the invention will be more fully understood from the following description of the invention, the referenced drawings attached hereto and the claims appended hereto.

To develop a new long-acting formulation of biopharmaceuticals such as peptide or protein the present invention relates to a composition for delivering a peptide comprising a conjugate of peptide with HA derivative, a method of preparing a conjugate of peptide with HA derivative, and a method of delivering a peptide comprising administering a conjugate of peptide with HA derivative to a subject in need.

The present invention will be described in detail.

One embodiment of the present invention provides a composition for delivering a peptide comprising a conjugate of peptide with HA derivative.

Peptide can be delivered using the compositions and methods of the specific embodiments of the present invention. If the peptide has thiol group at the end of the peptide sequence or can further comprise cysteine bonded to the end of the sequence, it is not limited. Suitable peptides for the use in the present invention include, but are not limited to, peptide which is an agonistic or antagonistic peptide for inflammatory disease associated with formyl peptide receptor like 1(FPRL1), and a agonistic or antagonistic peptide for diabetes associated with glucagon like peptide-1 (GLP-1). The agonistic peptide selectively binds to and activates FPRL1 receptor in FPRL1 over-expressing RBL-2H3 cells, and antagonistic peptide inhibits the binding of agonists to the specific receptor, FPRL1. More preferably, the agonistic or antagonistic peptide for inflammatory disease associated with FPRL1 is selected from the group consisting of WRYMVm (SEQ ID NO: 1), WKYMVm (SEQ ID NO: 3), WRWWWW (SEQ ID NO: 2), and wWRWWM (SEQ ID NO: 4).

In addition, protein also can be conjugated with HA derivative if the protein has thiol group at the end of the protein sequence or can comprise further cysteine bonded to the end of the protein sequence. The term “protein”, as used herein, refers to any therapeutic protein for administration to a subject, such as a human or other mammal. Suitable therapeutic protein for use include, but are not limited to, erythropoietin (EPO), interferon (IFN), monoclonal antibody-based product (Mab), insulin, fusion protein, and human growth hormone (hGH).

The molecular weight of HA is not limited, but preferably, can be 10,000 Da to 3,000,000 Da considering for use of delivery of protein.

And, the term “HA derivatives”, as used herein, refers to any HA derivatives, of which degradation in vivo is suppressed or regulated, linked by the conjugation with the peptide. In specific embodiment, the present invention provides a conjugate of HA derivatives by linking a peptide with HA derivative of which a terminal group is reacted with aminoethyl methacrylate (AEMA) or aminopropyl methacrylamide hyaluronic acid (HA-APMAm). The hyaluronic acid is modified with 2-aminoethyl methacrylate (AEMA) or N-(3-Aminopropyl) methacrylamide hydrochloride (APMAm) in an organic solvent to produce HA-AEMA or HA-APMAm. HA-AEMA or HA-APMAm is linked to a peptide containing thiol group with Michael addition to obtain a conjugate of HA derivative and a peptide.

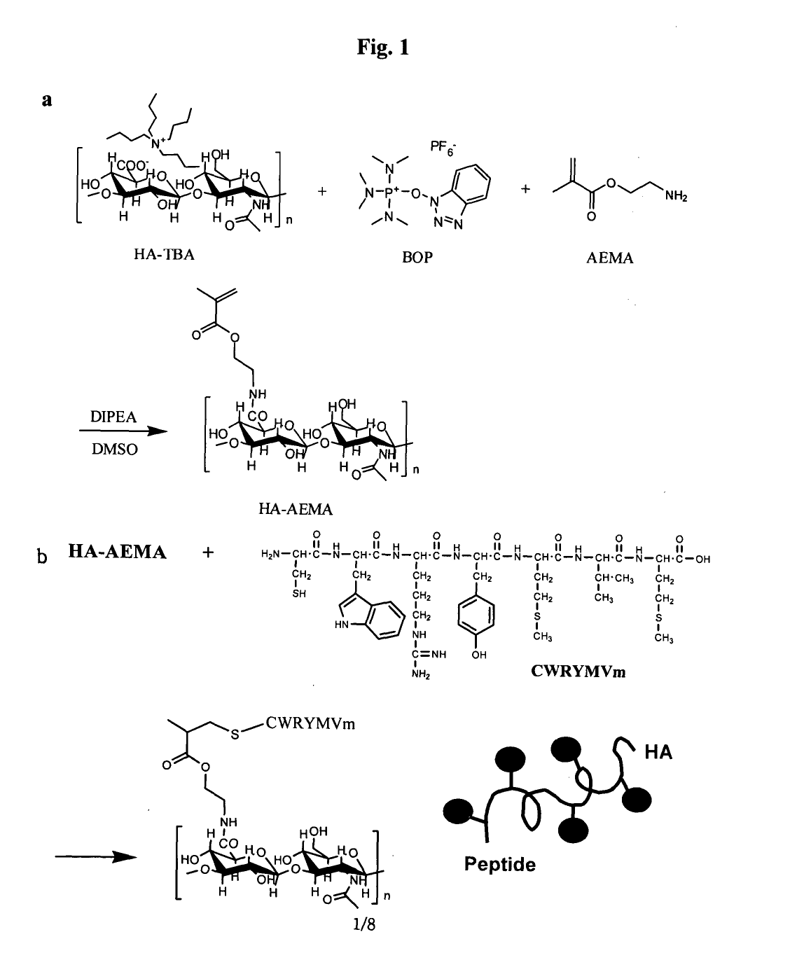

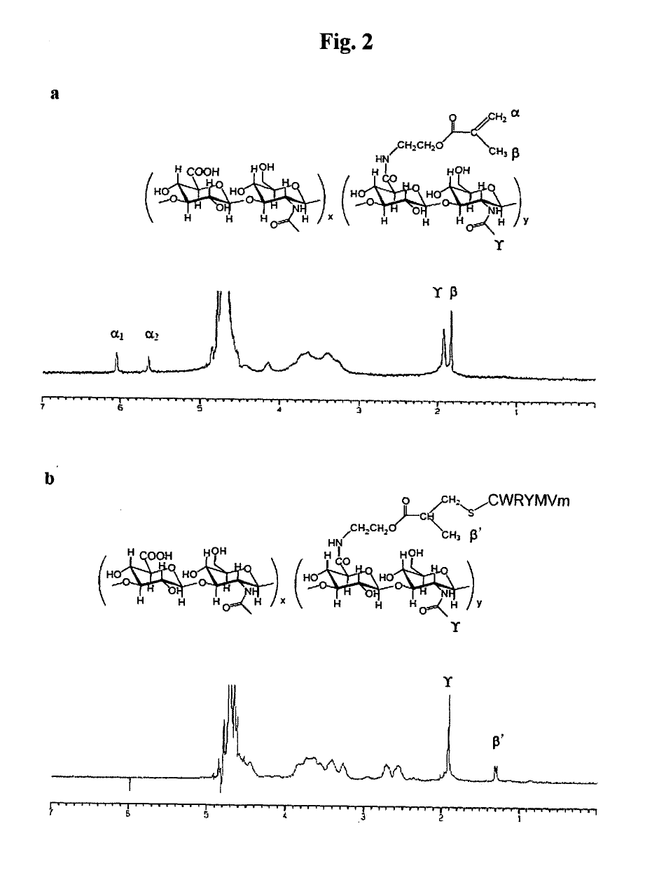

As shown in FIG. 1(a), HA-AEMA can be synthesized by the coupling reaction of tetrabutyl ammonium salt of HA (HA-TBA) and AEMA using benzotriazol-1-yloxy-tris(dimethyl-amino) phosphonium hexafluorophosphate (BOP) in DMSO, according to WO2008/048079. However, the preparation method of HA-AEMA is not limited to the above. The chemical structure of HA-AEMA prepared according to WO2008/048079 can be analyzed by ¹H NMR. The methyl resonance of acetamido moiety of HA at δ=1.85˜1.95 ppm can be used as an internal standard (γ in FIG. 2). By changing the amount of AEMA added for the reaction, the degree of AEMA modification in HA-AEMA can be controlled up to 85 mol %. Preferably the degree of AEMA modification in HA derivative can be controlled 10 to 70 mol %.

And HA-APMAm can be synthesized according to WO2008/048079. However, the preparation method of HA-APMAm is not limited to that and the chemical structure of HA-APMAm prepared according to WO2008/048079 can be analyzed by ¹H NMR.

And then, a peptide can be conjugated with HA derivative via Michael addition reaction between methacryloyl group of HA derivative and thiol group of the peptide. Therefore, if the peptide has no thiol group at the end of the peptide sequence, the peptide can be prepared by adding cytseine to the end of the peptide sequence before the Michael addition reaction.

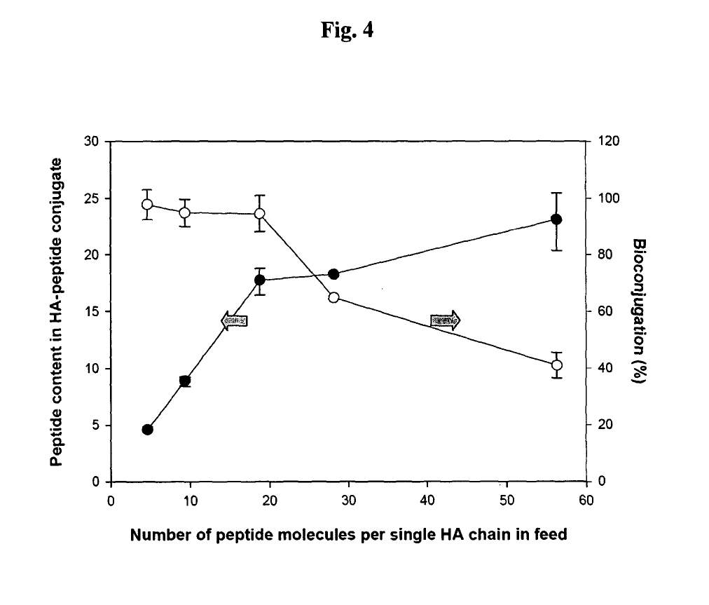

In addition, the average number of peptide molecules conjugated with the HA derivative could be controlled from 3 to 60 per single HA-AEMA chain. By conjugating many numbers of peptide molecules with the HA derivative per single HA derivative chain, it enables multiple actions of peptide drugs. The number of the conjugated peptide molecules could be controlled from 3 to 60 per single HA derivative chain, by adjusting the feeding rate of the peptide solution to the HA derivative. As shown in FIG. 4, if the number of peptide molecules per single HA-AEMA chain in feed is higher, the bioconjugation rate is lower. Therefore, considering peptide content in the conjugate and bioconjugation rate, the number of conjugated peptide molecules with HA derivative per single HA derivative chain can be preferably 5 to 30.

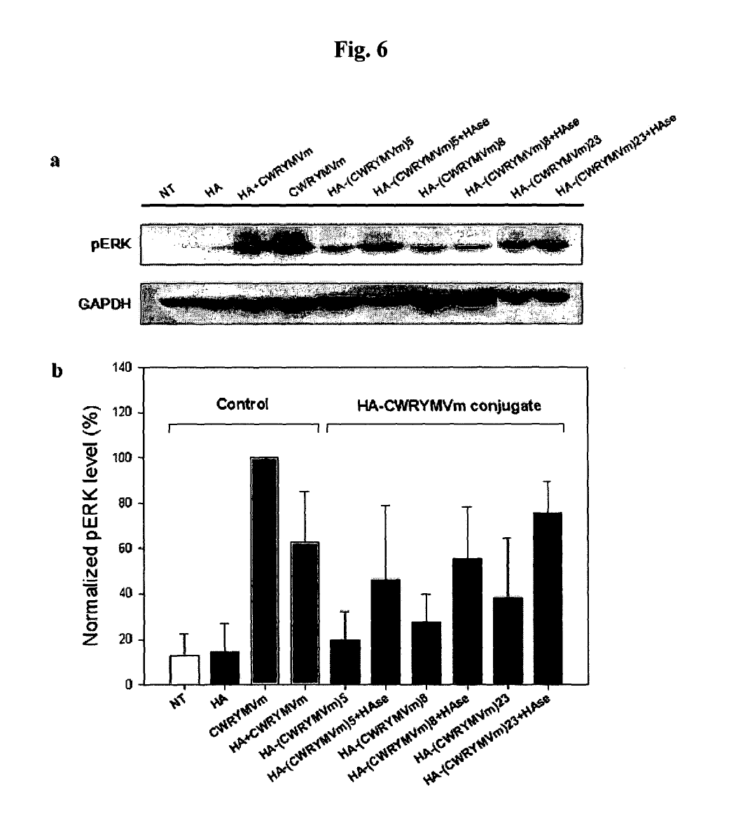

If the conjugate of HA derivative and a peptide is treated with hyaluronidase, the bioactivity of the conjugate is increased. The partially decreased biological activity of conjugate of HA derivative and peptide by the steric hindrance of HA can be recovered after its degradation by hyaluronidase treatment. As shown in FIG. 6b, the bioactivity could be recovered up to ca. 76% after degradation of HA molecules by hayluronidase treatment.

A biopharmaceuticals of the present invention, particularly peptide is referred to active or therapeutic material dissolved or dispersed in a pharmaceutically acceptable carrier or diluant in an effective amount. The carriers or diluents include any solvent, dispersing medium, coating, antibiotic agent, antifungal agent, isotonic agent, and absorption retarding agent, and the like. A supplemental active agent can be contained in the composition of the present invention.

The pharmaceutical composition can be prepared by a skilled person in this art in accordance with the general preparation method of pharmaceutical composition. In general, the composition can be formulated in forms of solution or suspension; injectable solution, solid formulation, or suspension; tablet, or solid formulation suitable for enteric administration; time release capsule; cream, lotion, salve, or inhalant.

The formulation can be administered in a pharmaceutically effective matter according to a general method. The formulation can be administered in various routes, for examples injection or capsule. The administration amount of active agent can be varied depending on the subject. The amount of active agent can be determined by a doctor.

Another embodiment of the present invention provides a method of preparing a conjugate of peptide with HA derivative comprising the steps of: synthesizing HA derivative, and mixing HA derivative and peptide solution comprising the peptide.

As set forth above, before mixing HA derivative and peptide solution, the peptide can be prepared by adding cysteine to the end of the peptide sequence in the case of the peptide has no thiol group at the end of the peptide sequence. And the conjugate is prepared by reacting between methacryloyl group of HA derivative and thiol group of the peptide.

In addition, the conjugate is prepared by reacting between methacryloyl group of HA derivative and thiol group of the protein. The protein may be selected from the group consisting of erythropoietin (EPO), interferon (IFN), monoclonal antibody-based product (Mab), insulin, fusion protein, and human growth hormone (hGH), but is not limited thereto. And the peptide can be selected from the group consisting of an agonistic or antagonistic peptide for inflammatory disease associated with formyl peptide receptor like 1 (FPRL1), and an agonistic or antagonistic peptide for diabetes associated with glucagon like peptide-1 (GLP-1). More preferably, the agonistic or antagonistic peptide for inflammatory disease associated with FPRL1 is selected from the group consisting of WRYMVm (SEQ ID NO: 1), WKYMVm (SEQ ID NO: 3), WRWWWW (SEQ ID NO: 2), and wWRWWM (SEQ ID NO: 4).

The HA derivative is not limited, if it can conjugate thiol group of the protein, and preferably can be aminoethyl methylated hyaluronic acid (HA-AEMA) or aminopropyl methacrylamide hyaluronic acid (HA-APMAm).

And the molecular weight of HA in the HA derivative is not limited but preferably can be 10,000 Da to 3,000,000 Da, considering for the use of delivering a drug. And the average number of peptide molecules conjugated with the HA derivative could be controlled from 3 to 60 per single HA derivative chain. By conjugating many numbers of peptide molecules with the HA derivative per single HA derivative chain, it enables multiple actions of peptide drugs.

The other embodiment of the present invention provides a method of delivering a peptide comprising administering a conjugate of peptide with HA derivative to a subject in need.

The peptide can be selected from the group consisting of an agonistic or antagonistic peptide for inflammatory disease associated with formyl peptide receptor like 1(FPRL1), and agonistic or antagonistic peptide for diabetes associated with glucagon like peptide-1 (GLP-1), but is not limited thereto. More preferably, the agonistic or antagonistic peptide for inflammatory disease associated with FPRL1 can be selected from the group consisting of WRYMVm (SEQ ID NO: 1), WKYMVm (SEQ ID NO: 3), WRWWWW (SEQ ID NO: 2), and wWRWWM (SEQ ID NO: 4).

The HA derivative is not limited, if it can conjugate thiol group of the peptide, and preferably can be aminoethyl methylated hyaluronic acid (HA-AEMA) or aminopropyl methacrylamide hyaluronic acid (HA-APMAm), but is not limited thereto.

And the molecular weight of HA in the HA derivative is not limited but preferably can be 10,000 Da to 3,000,000 Da, considering for the use of delivering a drug. And, the average number of peptide molecules conjugated with the HA derivative could be controlled from 3 to 60 per single HA derivative chain. By conjugating many numbers of peptide molecules to the HA derivative per single HA derivative chain, it enables multiple actions of peptide drugs.

The present invention is further explained in more detail with reference to the following examples. These examples, however, should not be interpreted as limiting the scope of the present invention in any manner.

### Example 1

### Preparation of Conjugate of Peptide with HA-AEMA for Inflammatory Disease Associated with FPRL1 Receptor

1.1. Materials

Sodium hyaluronate, sodium salt of hyaluronic acid (HA), with a molecular weight of 200,000 was obtained from Denkikagaku Kogyo Co. (Tokyo, Japan). Peptide with a sequence of Cys-Trp-Arg-Tyr-Met-Val-DMet (CWRYMVm, SEQ ID NO: 5) was purchased from Peptron (Daejeon, Korea). Dowex® 50WX8-40 ion-exchange resin, benzotriazol-1-yloxy-tris(dimethyl-amino)phosphonium hexafluoro-phosphate (BOP), 2-aminoethyl methacrylate hydrochloride (AEMA), N,N-diisopropylethylamine (DIPEA), tris(2-carboxyethyl) phosphine hydrochloride (TCEP), trifluoroacetic acid (TFA) and hyaluronidase from Streptomyces hyalurolyticus were purchased from Sigma-Aldrich (St. Louis, Mo., USA). Tetra-n-butylammonium hydroxide (TBA-OH) was obtained from Alfa Aesar (Ward Hill, Mass., USA). Dimethyl sulfoxide (DMSO) was obtained from Junsei Chemical Co. (Tokyo, Japan) and acetonitrile from J. T. Baker (Phillipsburg, N.J., USA). Anti-rabbit polyclonal antibody to phospho-ERK was purchased from Cell Signaling Technology (Danvers, Mass., USA) and anti-mouse monoclonal antibody to GAPDH from Biogenesis (Poole, UK). Goat anti-rabbit IgG or goat anti-mouse IgG antibody conjugated to horseradish peroxidase was obtained from KPL (Gaithersburg, Md., USA). Double distilled water was used for the following experiments. All chemicals were used without further purification.

1.2. Synthesis of HA-AEMA

Ion exchange resin of Dowex® 50WX-8-400 (25 g) was washed with 500 mL of water and filtered to remove the supernatant three times. Then, 1.5 molar excess of TBA-OH (48.9 mL) was added to the Dowex resin and mixed for 30 minutes. The filtered Dowex-TBA resin was washed with 500 mL of water and filtered again three times. Sodium salt of HA (M_{W}=200 K, 5 mmol) was dissolved in 200 mL of water, which was poured to the prepared Dowex-TBA (25 mmol) resin. After mixing for 3 hrs, the supernatant was filtered through 0.45 μm filter and lyophilized for 3 days. The resulting HA-TBA was dissolved in DMSO. Then, BOP, 2-AEMA and N,N-DIPEA were added to the solution and mixed for a day. Finally, the reaction product was dialyzed against a large excess amount of water and lyophilized for three days. The obtained HA-AEMA was characterized with ¹H nuclear magnetic resonance (NMR, DPX300, Bruker, Germany).

1.3. Conjugating Peptide to HA-AEMA

Agonistic peptide for FPRL1 receptor (CWRYMVm, SEQ ID NO: 5) was dissolved in water. For the reduction of disulfide bond between peptide molecules, 10-fold molar excess of TCEP as a reducing reagent was added to the peptide solution and mixed for 10 minutes. HA-AEMA was also dissolved in water. After complete dissolution, the HA-AEMA solution was mixed with the peptide solution. The number of peptide molecules per single HA chain in feed was 5, 9, 19, 28 and 56, respectively. After the pH of the reaction mixture was adjusted to 8.74 by the addition of 1 N NaOH, the mixed solution was incubated at 37° C. for a day. Then, the reaction was stopped by dropping the pH to 7.0 with 1 N HCl. The solution was finally lyophilized for three days. Conjugate of peptide with HA-AEMA was purified by the fractionation using gel permeation chromatography (GPC). GPC analysis was performed using the following systems: Waters 1525 binary HPLC pump, Waters 2487 dual λ absorbance detector, Waters 717 plus auto-sampler, Superdex Peptide 10/300 GL column. The eluent was 30 vol % acetonitrile (ACN)/0.1 vol % trifluoroacetic acid (TFA) and the flow rate was 1 mL/min. The detection wavelengths were 210 nm for HA and 280 nm for the peptide, respectively. Three replicates were carried out to assess the average peptide content in conjugate of peptide with HA derivative, and the bioconjugation efficiency (%).

The chemical modification of HA with AEMA was successfully carried out using the novel protocol as schematically shown in FIG. 1a. HA-AEMA was synthesized by the coupling reaction of TBA salt of HA (HA-TBA) with AEMA using BOP in DMSO. DIPEA was used to release the free primary amine of AEMA. The chemical structure of HA-AEMA was analyzed by ¹H NMR. The methyl resonance of acetamido moiety of HA at δ=1.85˜1.95 ppm was used as an internal standard (γ in FIG. 2). The degree of AEMA modification was ca. 57 mol %, which was determined from the peak areas of methacrylate unit of AEMA at S=6.1 and 5.6 ppm (α₁ and α₂ in FIG. 2a). By changing the amount of AEMA added for the reaction, we could control the degree of AEMA modification in HA-AEMA up to 85 mol %.

The peptide for FPRL1 receptor having a sequence of WRYMVm (SEQ ID NO: 1) was modified with cysteine to prepare CWRYMVm (SEQ ID NO: 5) with thiol groups. HA-AEMA was conjugated with CWRYMVm (SEQ ID NO: 5) via Michael addition reaction between methacryloyl groups in HA-AEMA and thiol groups in CWRYMVm (SEQ ID NO: 5) (FIG. 1b).

¹H NMR analysis of conjugate of peptide with HA derivative confirmed the formation of the conjugate (FIG. 2b). Before conjugation, there were double peaks at S=6.1 and 5.6 ppm corresponding to two hydrogens on methacryloyl groups of HA-AEMA (α₁ and α₂). After Michael addition reaction, the peaks at δ=6.1 and 5.6 ppm disappeared and the peak corresponding to —CH₃ of methacryloyl group at δ=1.8 ppm (β) was shifted to the peak at δ=1.3 ppm (β′). Although the methacryloyl groups of HA-AEMA are not completely consumed by Michael addition reaction, but the remained methacryloyl groups are removed by ‘deesterification’. The results from ¹H NMR analysis corroborated the successful formation of HA-AEMA and the subsequent conjugate of peptide with HA derivative.

### Example 2

### Quantification of Peptide Content in a Conjugate of Peptide with HA-AEMA for Inflammatory Disease Associated with FPRL1 Receptor

2.1. Quantification of Peptide Content in Conjugate of Peptide with HA-AEMA

A peptide stock solution at a concentration of 1 mg/mL was used to prepare peptide standard solutions with a concentration of 10, 20, 40, 80, 160 and 320 μg/mL, respectively. Conjugate of peptide with HA-derivative solutions were also prepared by dissolving 1 mg of each conjugate sample in 1 mL of water. GPC analysis was carried out as described above. From the peak areas detected at 280 nm, a linear standard curve for peptide was obtained and used for the determination of the amount of peptide in the conjugates.

2.2. Characterization of Conjugate of Peptide with HA-AEMA

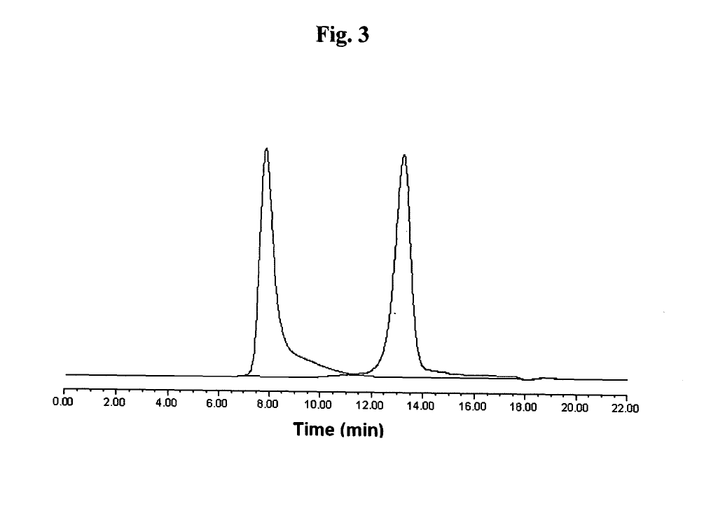

The formation of conjugate of peptide with HA-AEMA for FPRL1 receptor was also confirmed by GPC analysis as shown in FIG. 3. The peak of the conjugate appeared at a retention time of 8 min, while that of intact peptide appeared at a retention time of 13 minutes. The peak shift to the early retention time revealed that the peptide was conjugated to HA with a high molecular weight of 200,000 Da. The number of peptide molecules added per single HA chain in the reaction solution for the synthesis of the conjugate was 5, 9, 19, 28 and 56, respectively.

The resulting peptide content in the conjugates was quantified by measuring the peak area on GPC detected at 280 nm. Because HA is not detected at a wavelength of 280 nm, the peaks of the conjugates at 280 nm resulted solely from the peptide molecules. The peptide content in conjugates at a retention time of 8 min increased with the feeding ratio of peptide molecules to single HA derivative chain (FIG. 4). However, the degree of bioconjugation (%) decreased with increasing peptide content in the reactants. The bioconjugation (%) represents the molar ratio of peptide molecules in conjugate to the total peptide molecules added initially for the conjugation reaction. When the number of peptide molecules per single HA derivative chain in feed was less than 19, the bioconjugation efficiency (%) was higher than 90%. The average number of conjugated peptide molecules could be controlled from 3 to 60 per single HA chain, as schematically shown in FIG. 1b.

### Example 3

### In Vitro Serum Stability of Conjugate of Peptide with HA-AEMA for Inflammatory Disease Associated with FPRL1 Receptor

3.1. In Vitro Serum Stability Test of Conjugate of Peptide with HA-AEMA

In order to investigate the effect of HA conjugation on the serum stability of peptide, raw peptide and three kinds of conjugate samples were dissolved in 0.5 mL of water and mixed with 0.5 mL of fetal bovine serum (FBS), respectively. In conjugates of peptide with HA derivative, the number of peptide molecules per single HA chain was 5, 19, and 33, respectively. Because the peptide was not dissolved in serum completely, 50 vol % serum solution was used for the serum stability test. Then, the solutions were incubated at 37° C. for 96 hours. The remaining amount of peptide was measured by GPC analysis after incubation for 12, 24, 48, 72, and 96 hours. Three replicates were carried out.

3.2. In Vitro Serum Stability of Conjugate of Peptide with HA-AEMA

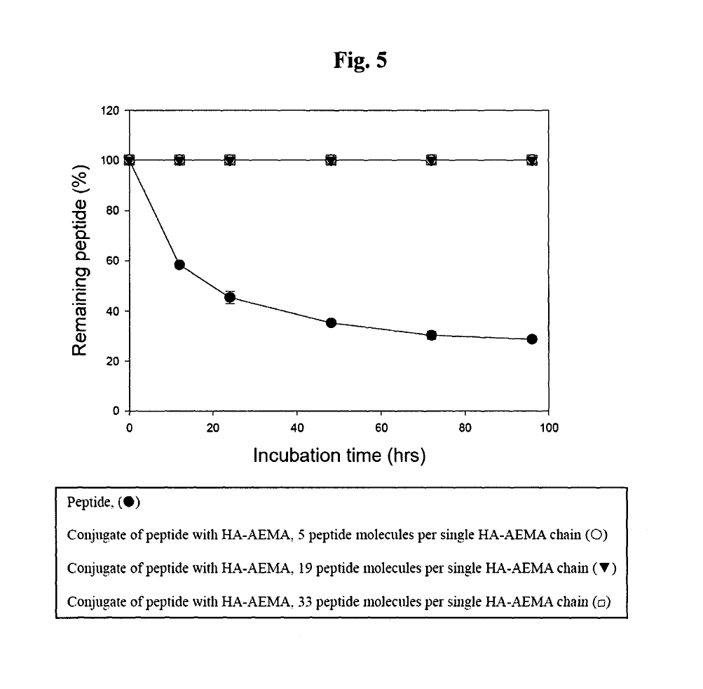

Although several agonistic and antagonistic peptides for FPRL1 receptor have been identified as promising new drug candidates for the inflammatory diseases such as sepsis, asthma and rheumatoid arthritis, their short half lives in the body should be elongated for further clinical applications. As expected, the peptide with a sequence of CWRYMVm (SEQ ID NO: 5) was quite unstable in serum. After incubation in 50 vol % FBS solution for 3 days, only less than 30 wt % of the peptide remained with a big initial degradation over 50 wt % in a day (FIG. 5). However, conjugate of peptide with HA derivative showed notably increased serum stability in 50 vol % FBS solution. The peptide conjugated to HA did not degrade at all even after incubation for 4 days. The increased stability of peptide in serum by HA conjugation was the same for all three kinds of conjugates samples with different peptide contents.

As the serum stability of peptide drug is essential for the application in vivo, the protocol for the conjugation of peptide to HA via Michael addition would be usefully exploited for the development of peptide therapeutics with a good solubility and a feasible long half-life.

### Example 4

### In Vitro Signal Transduction of Conjugate of Peptide with HA-AEMA for Inflammatory Disease Associated with FPRL1 Receptor

4.1. Hyaluronidase Treatment of Conjugate of Peptide with HA-AEMA

In order to investigate the effect of HA conjugation on the signal transduction activity of peptide, three kinds of samples were prepared, the raw peptide, conjugate of peptide with HA-AMEA, and conjugate of peptide with HA-AEMA after hyaluronidase treatment. In the conjugate samples, the number of peptide molecules per single HA derivative chain was 5, 8, and 23, respectively. Each sample of conjugate of peptide with HA-AEMA (0.4 mL, 1 mg/mL) was divided into two aliquots. One aliquot was mixed with 0.2 mL of hyaluronidase solution (400 units/mL) and the other was mixed with 0.2 mL of water. Then, the solutions were incubated at 37° C. overnight for the complete enzymatic degradation of HA. Three replicates were carried out for the following bioactivity tests measuring the intracellular level of pERK and calcium ion.

4.2. RBL-2H3/FPRL1 Cell Culture for Western Blot

FPRL1 over-expressing RBL-2H3 (RBL-2H3/FPRL1) cells were cultured at 37° C. in a humidified incubator containing 5% CO₂, as described in He et al., Blood 101, 1572-1581, 2003. Dulbecco's modified Eagle's medium (DMEM) was supplemented with 20 vol % heat-inactivated FBS and 200 μg/mL of G418. The cells were sub-cultured every 3 days.

4.3. Stimulation of RBL-2H3/FPRL1 Cells with Peptide Samples

The prepared cells were aliquoted into 1×10⁶ cells and stimulated with a control (no treatment), peptide (CWRYMVm, SEQ ID NO: 5), HA-AEMA, the mixture of peptide and HA-AEMA, and three kind of conjugate samples with and without hyaluronidase treatment. For comparison, the peptide content in conjugate samples was adjusted to have the same amount with the peptide sample. The stimulation time varied from 0 to 30 minutes. After stimulation, the cells were washed with serum-free DMEM and lysed in lysis buffer at pH=7.4 containing 20 mM Tris-HCl, 150 mM NaCl, 1 wt % Triton X-100, 50 mM NaF, 1.5 mM Na₃VO₄ and 1 mM phenylmethylsulfonyl fluoride. The detergent-insoluble material was pelleted by centrifugation at 12,000 g and 4° C. for 15 min, and the soluble supernatant fraction was used immediately or stored at −80° C. before use. Protein concentrations in the lysates were determined by Bradford protein assay. Three replicates were carried out.

4.4. Electrophoresis and Immunoblot Analysis for Western Blot

Protein samples were subjected to electrophoresis using 12 wt % SDS-polyacrylamide gel and the buffer system described by King et al. J. Mol. Biol. 62, 465-477, 1971. After the electrophoresis, the proteins were blotted onto nitrocellulose membrane and blocked by incubating with Tris-buffered saline, 0.05% Tween 20 containing 5% nonfat dried milk. Then, the membranes were incubated for 12 hrs with anti-rabbit polyclonal antibody to pERK (1/1000 dilution) or anti-mouse monoclonal antibody to GAPDH (1/2000 dilution) and washed with Tris-buffered saline. After incubating the membrane with a 1/5,000 diluted goat anti-rabbit IgG or goat anti-mouse IgG antibody conjugated to horseradish peroxidase for 1 hr, the antigen-antibody complexes were visualized by using the enhanced chemi-luminescence (ECL) detection system.

4.5. Calcium Imaging of the Cells

The intracellular calcium level was determined using fura-2-acetoxymethyl ester (Fura-2/AM) as described by Bae et al., Blood 97, 2854-2862, 2001. Briefly, the prepared cells were incubated in serum-free RPMI 1640 medium with 3 μM of Fura-2/AM at 37° C. for 50 min under continuous stirring. For each measurement, 2×10⁶ cells were aliquoted in Ca²⁺-free Locke's solution (154 mM NaCl, 5.6 mM KCl, 1.2 mM MgCl₂, 5 mM HEPES, pH 7.3, 10 mM glucose, and 0.2 mM EGTA). Changes in the fluorescence ratio were measured at the dual excitation wavelengths of 340 nm and 380 nm, and the emission wavelength of 500 nm, and the fluorescence images were obtained.

4.6. Assessment of Antagonistic Peptide for FPRL1 Receptor

In order to investigate the effect of co-treatment with antagonistic peptide (C—WRWWWW (SEQ ID NO: 6), WRW4 (SEQ ID NO: 2) on the binding characteristics of the conjugates of peptide with HA-AEMA to FPRL1 receptor, the RBL-2H3/FPRL1 cells were stimulated with a control (no treatment), peptide (CWRYMVm, SEQ ID NO: 5), and three kinds of conjugate samples for 5 minutes in the presence and absence of C-WRWWWW (SEQ ID NO: 6), (WRYMVm, SEQ ID NO: 1, competitor for FPRL1 binding). The other procedures including electrophoresis and immunoblot analysis for Western blot was repeated in the same way as described above.

4.7. Statistical Analysis

The data are expressed as means±S.D. from several separate experiments. Statistical comparisons were conducted via Student's t test, and a value for p<0.05 was considered statistically significant.

4.8. In Vitro Signal Transduction of Conjugate of Peptide with HA-AEMA

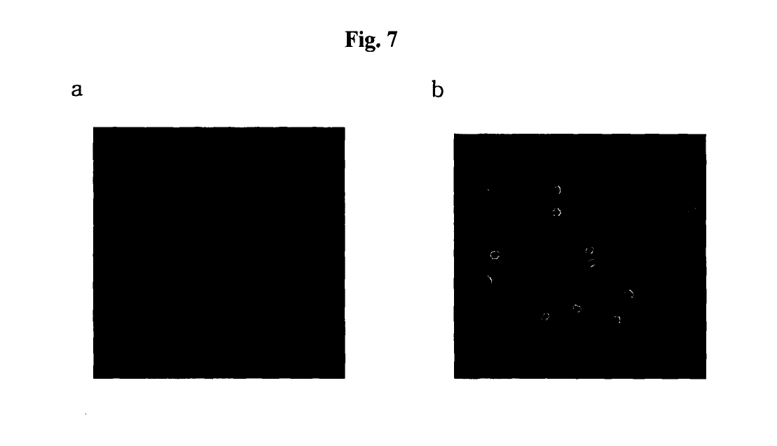

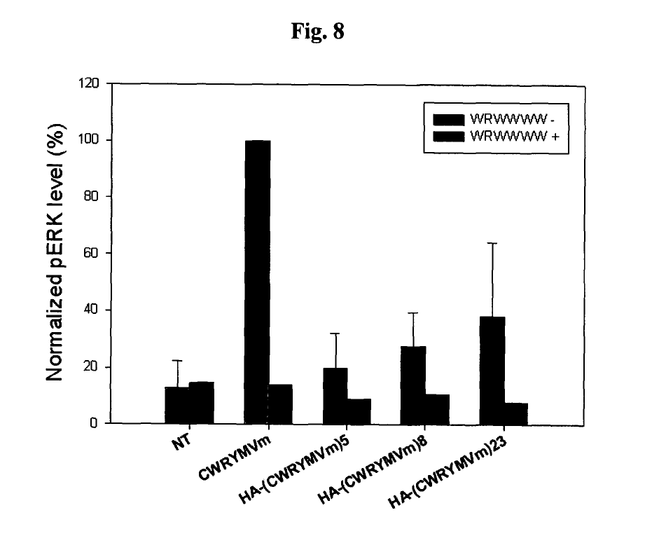

The signal transduction activity of the conjugate for FPRL1 receptor was assessed by measuring the elevation level of phospho-extracellular signal-regulated kinase (pERK) (FIGS. 6 and 8) and calcium ion (FIG. 7) in RBL-2H3/FPRL1 cells. The band intensity was normalized to GAPDH to get the pERK level. When the cells were stimulated with the peptide or conjugate for FPRL1 receptor up to 30 min, the elevation of intracellular pERK level on Western blot was the highest at a stimulation time of 5 min in both cases. Therefore, the stimulation time was fixed at 5 min for the following cell activity tests. FIG. 6a shows the pERK levels on Western blots after stimulation with a control (no treatment), HA-AEMA (HA), peptide (CWRYMVm, SEQ ID NO: 5), a mixture of HA-AEMA and peptide (HA+CWRYMVm), conjugate of peptide with HA-derivative (HA-CWRYMVm), and hyaluronidase treated conjugate of peptide with HA-derivative (+HAse). The numbers (5, 8, and 23) represent the number of peptide molecules per single HA derivative chain in the conjugates.

As a control, the pERK level by peptide alone was set to be 100% and the other data were normalized for comparison. In the mixture of HA-AEMA and peptide, the pERK level decreased to ca. 63% compared with that of peptide alone. Moreover, the bioactivity of the conjugate of peptide with HA-AEMA decreased to ca. 20%, 28%, and 38% depending on the amount of peptide per single HA chain. However, the bioactivity could be recovered up to ca. 76% after degradation of HA molecules by hyaluronidase treatment (+HAse).

The decrease in the signal transduction activity of peptide molecules after being conjugated with HA might be resulted from the ‘steric hindrance’ of HA chain reducing the access of peptide molecules to FPRL1 receptor. Due to the same reason, conjugate of peptide with HA derivative with a low peptide content of 5 per single HA chain [HA-(CWRYMVm)5] showed a lower pERK level than HA-(CWRYMVm)23, despite of using the same amount of peptide content for the Western blot analysis.

After the hyaluronidase treatment (+HAse), however, the steric hindrance was thought to be alleviated contributing for the increase of intracellular pERK level. As well as the result without hyaluronidase treatment, there was positive correlation between the pERK level and the content of peptide in conjugate of peptide with HA derivative after hyaluronidase treatment. The p-value between HA-AEMA (HA) and [HA-(CWRYMVm)5+HAse] was 0.194, which means no significant elevation of pERK level after treatment with [HA-(CWRYMVm)5+HAse]. However, as the content of peptide in the conjugates increased to 9 and 23, the p-value decreased to 0.056 and 0.004, respectively. The decreased p-value indicates that the biological activity of HA-CWRYMVm after hyaluronidase treatment significantly increased with the peptide content in HA-CWRYMVm conjugates.

Calcium imaging showed the same tendency with the Western blot analysis. Unlike the control, the RBL-2H3/FPRL1 cells treated by conjugate of peptide with HA derivative showed a bright fluorescence under the UV light demonstrating its biological activity (FIG. 7). On the other hand, when the cells were co-treated with antagonistic peptide (WRWWWW, SEQ ID NO: 2) for FPRL1 receptor, the intracellular level of pERK did not increase at all (FIG. 8). The antagonistic peptide down-regulates the activation of FPRL1 by agonistic peptide, resulting in the complete inhibition of the intracellular calcium increase, extracellular signal-regulated kinase activation, superoxide generation, and chemotactic migration of cells toward agonistic peptides.

All together with the stimulation test results of RBL-2H3/FPRL1 cells with agonistic and antagonistic peptides, the conjugate of peptide with HA derivate of the present invention shows the signal transduction activity of the conjugates for FPRL1 receptor. The conjugation of HA-AEMA with the peptide drug for FPRL1 receptor would be usefully applied for further in vivo applications. In addition, HA derivative would be used as a novel drug carrier for the conjugation of various protein and peptide drugs with thiol groups.

What is claimed is:

1. A composition for delivering a peptide comprising a conjugate of peptide with hyaluronic acid(HA) derivative.

2. The composition according to claim 1, wherein the peptide is selected from the group consisting of an agonistic or antagonistic peptide for inflammatory disease associated with formyl peptide receptor like 1(FPRL1), and agonistic or antagonistic peptide for diabetes associated with glucagon like peptide-1 (GLP-1).

3. The composition according to claim 2, wherein the agonistic or antagonistic peptide for inflammatory disease associated with FPRL1 is selected from the group consisting of WRYMVm (SEQ ID NO: 1), WKYMVm (SEQ ID NO: 3), WRWWWW (SEQ ID NO: 2), and wWRWWM (SEQ ID NO: 4).

4. The composition according to claim 1, wherein the peptide can comprises further cysteine bonded to the end of the peptide sequence or has thiol group at the end of the peptide sequence.

5. The composition according to claim 1, wherein the hyaluronic acid has molecular weight of 10,000 Da to 3,000,000 Da.

6. The composition according to claim 1, wherein the HA derivative is aminoethyl methylated hyaluronic acid (HA-AEMA) or aminopropyl methacrylamide hyaluronic acid (HA-APMAm).

7. The composition according to claim 1, wherein the average number of the peptide molecules conjugated with the HA derivative is 3 to 60 per single HA derivative chain.

8. A method of preparing a conjugate of peptide with HA derivative comprising the steps of:
synthesizing HA derivative; and
mixing HA derivative and peptide solution comprising the peptide.

9. The method of preparing a conjugate of peptide with HA derivative according to claim 8, wherein, before mixing HA derivative and peptide solution, the peptide is prepared by adding cysteine to the end of the peptide sequence in the case of the peptide has no thiol group at the end of the peptide sequence.

10. The method of preparing a conjugate of peptide with HA derivative according to claim 8, wherein the conjugate is prepared by reacting between methacryloyl group of HA derivative and thiol group of the peptide.

11. The method of preparing a conjugate of peptide with HA derivative according to claim 8, wherein the peptide is selected from the group consisting of an agonistic or antagonistic peptide for inflammatory disease associated with formyl peptide receptor like 1(FPRL1), and agonistic or antagonistic peptide for diabetes associated with glucagon like peptide-1 (GLP-1).

12. The method of preparing a conjugate of peptide with HA derivative according to claim 11, wherein the agonistic or antagonistic peptide for inflammatory disease associated with FPRL1 is selected from the group consisting of WRYMVm (SEQ ID NO: 1), WKYMYm (SEQ ID NO: 3), WRWWWW (SEQ ID NO: 2), and wWRWWM (SEQ ID NO: 4).

13. The method of preparing a conjugate of peptide with HA derivative according to claim 8, wherein the hyaluronic acid has molecular weight of 10,000 Da to 3,000,000 Da.

14. The method of preparing a conjugate of peptide with HA derivative according to claim 8, wherein the HA derivative is aminoethyl methylated hyaluronic acid (HA-AEMA) or aminopropyl methacrylamide hyaluronic acid (HA-APMAm).

15. The method of preparing a conjugate of peptide with HA derivative according to claim 8, wherein the average number of the peptide molecules conjugated with the HA derivative is 3 to 60 per single HA derivative chain.

16. A method of delivering a peptide comprising administering a conjugate of peptide with HA derivative to a subject in need.

17. The method according to claim 16, wherein the peptide is selected from the group consisting of an agonistic or antagonistic peptide for inflammatory disease associated with formyl peptide receptor like 1(FPRL1), and an agonistic or antagonistic peptide for diabetes associated with glucagon like peptide-1 (GLP-1).

18. The method according to claim 17, wherein the agonistic or antagonistic peptide for inflammatory disease associated with FPRL1 is selected from the group consisting of WRYMVm (SEQ ID NO: 1), WKYMVm (SEQ ID NO: 3), WRWWWW (SEQ ID NO: 2), and wWRWWM (SEQ ID NO: 4).

19. The method according to claim 16, wherein the molecular weight of the hyaluronic acid is 10,000 Da to 3,000,000 Da.

20. The method according to claim 16, wherein the average number of the peptide molecules conjugated with the HA derivative is 3 to 60 per single HA derivative chain.
